(19) United States
(12) Patent Application Publication
(10) Pub. No.: US 2016/0313236 A1
(43) Pub. Date: Oct. 27, 2016

(54) METHOD FOR DETERMINING THE CONCENTRATION OF A SUBSTANCE IN A DEFORMABLE CONTAINER

(71) Applicant: LASER- UND MEDIZIN-TECHNOLOGIE GMBH BERLIN, Berlin (DE)

(72) Inventors: Jürgen HELFMANN, Klein-Machnow (DE); Ingo GERSONDE, Berlin (DE); Hans-Joachim CAPPIUS, Berlin (DE); Karsten LIEBOLD, Ludwigsfelde (DE); Uwe NETZ, Berlin (DE)

(73) Assignee: LASER- UND MEDIZIN-TECHNOLOGIE GMBH BERLIN, Berlin (DE)

(21) Appl. No.: 14/902,458

(22) PCT Filed: Jul. 2, 2014
(86) PCT No.: PCT/EP2014/064111

§ 371 Date: Dec. 31, 2015

(30) Foreign Application Priority Data
Jul. 2, 2013 (DE) 10 2013 011 495.0

(51) Int. Cl. G01N 21/31; C12M 1/34; C12M 1/00; G01N 21/59; G01N 21/03

(52) U.S. Cl. CPC G01N 21/31; G01N 21/59; G01N 21/03; C12M 23/28; C12M 23/26; C12M 41/46; G01N 2021/0364; G01N 2201/062

(57) ABSTRACT

The invention relates to a non-destructive and non-invasive method for determining the concentration or other parameters of constituent substances in fluids, which method is capable of minimizing the optical interfering influences, which are unknown but constant during the individual measurement, of the vessel wall on the measurement result or the evaluation, in that measurements are carried out with different through-radiation path lengths and quotient calculations eliminate the influences of the vessel wall. Wide-area illumination and detection ensure that non-linearities occurring during said measurements do not interfere with the accuracies of the determination.

### OBJECT

The object of the invention is to optically determine the concentration of a substance in a container with a flexible walling, without having to separately detect the optical properties of the walls.

### STATE-OF-THE-ART

It is often necessary to determine parameters such as the concentration of one or more substances of samples or fluids in closed containers without opening the containers and withdrawing liquids. This includes fluids that are enclosed sterilely or fluids that strongly react to the surrounding milieus.

When the containers have partially transparent walls, spectroscopic methods can be used, which utilize the absorption and/or scattering caused by the substance to determine the desired parameters. Usually measurements for determining the concentration are performed at two wavelengths—one which is strongly dependent on the concentration of the substance, and a second one which is only weakly dependent on the concentration of the substance and serves for correction of other occurring weakenings. Other substances to be determined then require other wavelengths. This works well under identical measuring conditions, i.e., for example when the containers show a similar optical weakening.

These methods however become inaccurate when the relevant optical properties of the walls of the respective container are unknown, because the material, the wall thickness and the surface structure vary from one measuring situation to another.

A further problem arises when the containers or the contained substances scatter optical radiation. Methods are known for shining light through fluids and solids, which based on the length of the illumination pathway, knowledge of the input intensity and the molecular extinction of the substance allow determining the concentration of the contained substance from measurement of the starting intensity (Lambert Beer's law). Such methods have limits when the scattering of the container wall or the scattering of the contained substances are of similar magnitude (concentration-dependent) as the absorption of the inputted optical radiation.

Solutions are offered by methods which involve detecting non-absorbed radiation components, for example transverse to the direction of propagation, in order to determine the multiply scattered components.

Another known solution for multiple scattering occurring in the beam path is also to simulate the radiation propagation by means of theoretical assumptions (for example Monte Carlo simulation). This requires, however, that the measured properties of the samples to be measured are only caused by the concentration differences of the contained substances, or the optical properties of the container walls are known.

Also, when the optical properties of the container walls are known, a calibration by way of measurings with predetermined concentrations of the contained substances can be performed. Hereby a change of the measuring situation—i.e., the absorption and/or scattering of the container wall at the used radiation—leads to errors in the determination of the concentration of the contained substance.

These methods are classically performed at low scattering in the transmission path in a transmission arrangement, wherein the sample is positioned between the light source and the detector. An alternative in the case of more strongly scattering samples is the remission measuring, in which the transmission paths between the light source and the detector, which are positioned on the same side of the sample, traverse a volume by scattering determined by the scattering.

All these methods fail when the fluids are not provided in containers whose walls do not have the same properties, or the fluids cannot be transferred, for example because the containers of fluids originate from different manufacturers.

The reference DE 603 12 737 T2 describes a method derived from the scattered light measurement, in which a device and a method is described for detecting at least twice-scattered light with at least one LED as source (arranged as “group” about the circumference) and one or multiple photodiodes as receiver (also arranged as “group” about the circumference). This can be complemented by a circle of detectors, which are arranged at a greater distance along the tube. By detecting the scattering at infrared light without direct radiation from the LED onto the detector, the hematocrit is determined at a supply tube to the dialysis device by clamping in the tube. In order to be able to manage the calibration,—which is not mentioned but required—the tube line is clamped, i.e., the round tube geometry is changed into a surface, which is planar relative to the light source and the detector. The detection is performed with at least two light paths, i.e., an LED and at least two photodiodes arranged offsets to each other.

The reference DE 698 35 142 T2 describes a solution for measurement on plasma bag systems, wherein an automatically loadable tube holder is described to which a broadband light source and a spectrometer are connected with optical waveguides in transmission or reflection arrangement, wherein the radiation is transmitted through print that may be on the tube. From the slope of the weakening, which is measured at least at two wavelength ranges (by taking the lamp spectrum from a reference path into account) concentrations of hemoglobin, bilirubin, biliverdin, methylene blue (used for virus deactivation) and turbidity in intralipid-equivalents are determined.

When the remission measurement is used with different distances of the light source and the detector, the volume traversed by radiation can be influenced in a targeted manner and in this way a wall can be separated from the sample/fluid arranged there behind. This fails when the surface morphology (structure) of the wall causes scattering, is laterally different in the region of the measuring fields and/or the thickness of the wall is not adjusted to the light path so that no or only insufficient amounts of radiation enter the contained substance.

A method is therefore desirable, which enables minimizing optical interferences of the container wall with the measuring result or the analysis, which optical interferences are unknown, but are constant during the individual measurement.

From DE 198 80 369 a method is known for noninvasive in vivo determination of blood components by means of measuring light absorption while externally mechanically influencing the measured body part, which is impinged with two pressure modulation frequencies and is radiated with light of at least two wavelengths of which one, but not all, are within the range of optical absorption of the blood component. At least 4 measurement signals are obtained, which depend on the effect of the light as well as on the change in thickness, and from which the concentration of the blood component is determined. Hereby the change in thickness is caused by harmonic vibrations and is isolated from the measurement signal via the thickness modulation frequencies and is further processed. One of the frequencies can be zero—the other one should then no longer compress the blood-filled vessels, but only the tissue. With this the “interference” is principally isolated by the weakening in the tissue—i.e., it is filtered out via the wavelength change frequency—and the variable to be determined is measured as in blood contained in a cuvette. As measurement methods absorption measurements in transmission arrangement or remission arrangement at at least 2 wavelengths and acoustical-optical measurements are mentioned.

### SOLUTION ACCORDING TO THE INVENTION

According to the invention the object is solved by performing appropriate spectroscopic measurements at different transmission path lengths through the analyzed medium. As a result of the flexibility of the container it is possible to perform a transmission measurement through the container so that measurement results can be recorded in dependence on multiple different transmission path lengths. Hereby the variation of the distance can then be realized manually or also motorically automated. Defined distance but also a continuous distance change while continuously measuring can be analyzed. Because the particularly relevant geometry of the front and rear container wall does not change, however, but the liquid arranged therebetween does change, the change of the measurement values occurring hereby is influenced to a stronger decree by the interaction in the liquid located therebetween then by the container wall. Therefore an appropriate analysis enables minimizing the influences of the container wall. Suited analysis methods include for example multivariate data analyses. In simple cases, for example in the case of low optical scattering, a simple regression may be sufficient.

The object is also solved by a device or measuring device for optical noninvasive determination of the concentration or other parameters of contained substances in a flexible container with a light source, at least one detector and a processor. The light source is configured to shine light onto the flexible container, whereby the light is transmitted through the flexible container. The at least one detector is configured for each parameter to be detected to perform at at least one wavelength or a wavelength range the detection of the contained substance the weakening of the used radiation at different transmission path lengths. The processor is configured to eliminate the influence of the concrete container wall by forming a quotient of the measurements of different thicknesses. Essentially two configurations for the light source and the at least one detector of the device are possible. In a first configuration the device can have a light source, for example a light diode or multiple light diodes, which shines radiation with predetermined wavelengths or predetermined wavelength ranges onto the flexible container. In this case the at least one detector is configured to detect these predetermined wavelengths of predetermined wavelength ranges. For this purpose the at least one detector can for example be a wavelength-integrating sensor. In the second configuration, the device can have a broadband light source, which shines onto the flexible container with a continuous spectrum. In this case the at least one detector is configured for wavelength-resolving detection, for example with one or multiple spectrometers, monochromators or filters. The two configurations mentioned above can also complement each other or can be combined with each other. Thus for example a wavelength, which can be adjusted on the light source, can be shone onto the flexible container, and then reach a detector which can be adjusted to defined wavelengths, i.e., in this case the light source or light sources and also the detector or detectors can be adjusted to predetermined wavelengths or wavelength ranges. Therefore also multiple light sources and detectors can be arranged in parallel, in order to detect or determine multiple parameters in parallel.

### DETAILED DESCRIPTION OF THE INVENTION

Regarding the measuring device, continues spectra with broadband light sources can be used for the irradiation as well as radiation with a few selected wavelengths, which are for example realized with different light emitting diodes. In the first case the detection is performed wavelength-resolved with spectrometers, monochromators or filters. When irradiating with a few wavelengths or appropriate wavelength ranges, a wavelength integrating sensor can be used.

The parameter to be determined (i.e., the parameter whose value is to be determined) has to be suited for the optical detection (by spectroscopy), i.e., there have to be wavelengths or wavelength ranges at which a weakening of the radiation depends on the parameter to be determined of the contained substance(s).

The container has to have at least one region, which is transparent in this wavelength range; in the case of transmission measurements these have to be at least two opposing regions.

For a remission measurement the transparent region has to be sufficiently large, so that the remission is spatially resolved with beam propagation through the wall up to the region of the contained substances. This depends on the thickness and scattering of the wall and the scattering and absorption of the contained substances in the container. The wall has to have sufficiently similar optical properties over this measurement range.

In an embodiment the light source is arranged relative to the at least one detector so that the detected radiation traverses differently long paths through the contained substances in the container.

Hereby for each of the parameter to be detected of the contained substances, at least one wavelength has to be able to be analyzed. When unknown weakenings are based on other contained substances, at least one further wavelength is required.

The measurement is performed as intensity measurement at at least two transmission path lengths and respectively for the required wavelength or wavelength ranges. Hereby only one calibration with different walls of the containers is required a priori between the parameter to be determined and the intensity. This selection of the wall however does not have to be complete.

It is helpful to know the characteristic of the measurement arrangement (for example weakening as a result of thickness-dependent overradiation of the detector surface). Usually for the accurate determination of the parameters of the contained substance(s) with the used detector, a dark measurement (without illumination) is performed in order to determine the signal background, which can be subtracted from the determined measurement values. With this, systematic errors are reduced and accuracies of the parameter determination improved.

Knowledge regarding the walls of the containers is not required for the concrete measurement. The wall of course has to be permeable for the used wavelengths or wavelength ranges, because otherwise no radiation would reach the contained substances.

A measurement of the intensity of the light source at the required wavelength or the wavelength range prior to the measurement (without sample) enables a normalizing and thus a comparison of different measurements. An assumption of the intensity of the light source (instead of the measurement) at the used wavelength or the integral over the used wavelength range can replace a measurement, however, with lower staring certainty with regard to the analysis.

The measurement can also include a multiple reference measurement of the radiation intensity of the light source, in order to detect and compensate fluctuations of the light source.

The measurement data are analyzed by way of an response function R, which calculates the parameters to be determined from the measurement values (weakened intensity at one thickness an wavelength or a wavelength range Iₓ(□₁, dₓ), thickness of the container through which radiation is transmitted dₓ). Hereby the index x stands for a not further defined number of measurements (at least two) and □₁ for a wavelength or a wavelength range at which the parameter(s) to be determined of the contained substance are analyzable. This response function R is determined as follows:

When an irradiation and detection surface is selected, which is broad relative to the transmission path length, a one-dimensional radiation transport model can be applied. Hereby the angular distribution of the radiation when traversing the wall and the content substances is essentially important. The angular distribution is to be as constant as possible for the measurement. This can be accomplished by a sufficiently high scattering or thickness of the transmission path length through the contained substances or by a corresponding selection of the irradiation and detection surface. Thus beside the broad irradiation and detection surface also a minimal thickness of the fluid with the contained substances can be provided.

In an embodiment of the device the light source is configured for a wide-area irradiation and the detector for a wide-area detection.

The measured weakened intensity (Iₓ(ε₁,) is composed of a product of the contributions of first wall (W), second wall (W′) which can also be identical to the first wall, and sample/contained substance (P), i.e.,: W*P(d)*W′. For the analysis, the measurement values for the at least two thicknesses of the container to be measured are related to each other, i.e., divided by each other. Because the contributions of the wall W or W′ do not change with the change of thickness, the following relationship results:

$${Q}_{x 1} ( {λ}_{1} ) = \frac{W \cdot {W}^{′} \cdot P ( {d}_{x} )}{W \cdot {W}^{′} \cdot P ( {d}_{1} )} = \frac{P ( {d}_{x} )}{P ( {d}_{1} )}$$

More than one measuring value with the container thickness dx (x>1) can be determined at different time points, in order to increase the accuracy.

The thus canceled out contributions W and W′ of the wall to the weakening surprisingly have no remarkable influence on the accuracy of the determined parameters of the content substances, even though these values are measured.

The response function R can be generated in that the parameters to be determined of the contained substances are measured with the measurement values in a calibration measurement and according to the state of the art corresponding predictive functions are formed via a linear or non-linear regression by using the quotient formation according to the invention. In the case of greater scattering values this occurs in the weakening rather with non-linear regression models because a linear relationship is not necessarily given.

In an embodiment the device is configured to determine blood parameters, for example the hemoglobin content of blood, which is arranged in a closed blood conserve. The device can have a mechanical arrangement, which is configured to change the distance between two walls of the blood conserve in the region of a measuring field during the measurement in a defined manner.

A preferred application of the invention is therefore the determination of blood parameters on closed blood conserves, which after being opened or removed may no longer be infused due to the contamination risk. Depending on the manufacturer the blood bags used as container are different regarding the material, thickness and surface structure, which have an effect on the spectroscopy which is otherwise well suited for determining the relevant blood parameters.

By means of the mechanical arrangement, which changes the distance between the two walls of the blood bag in the region of a measurement field during the measurement in a defined manner, blood parameters such as for example the hemoglobin content can be determined. DE 698 28 825 T2 for example discloses that commercial devices exist in which such a determination is desired; the volume with confirmed concentration can also be expressed by a total amount of hemoglobin.

The invention further includes using the device for determining blood parameters of blood, which is arranged in a closed blood conserve.

A further preferred application is the use in disposable bioreactors, such as the wave bioreactor of GE Healthcare and the Biostat Cultibag RM of Sartorius Stedim Biotech, as well as the flexible bag systems of S.U.B, the Biostat Cultibag STR and the XDR Bioreactor and others. The measurement and control of the cell culture process in a disposable bioreactor is challenging because the plastic bag in which the cultivation takes place is a closed sterile system, the convectional sensors for process control, such as thermostat sensors, pH and conductivity measurement electrodes, glucose and oxygen electrodes, pressure sensors etc. cannot simply be introduced into the bag when needed but have to be already integrated in the sterile bag. This poses problems because on one hand the containers have to be produced, stored and shipped in a dry state and on the other hand further calibrations prior to use of the bag are not possible. Also it must be decided during the production of the systems what configuration of the possible sensors should be installed. The use of optical sensors can circumvent these problems because easy-to-integrate sensor materials (optical indicator materials) can be incorporated cost-effectively and can be used with the required readout technology if needed. Usually the variables to be determined include the cell number, which due to its turbidity, which increases with cell growth, complicates measurements. Here a thickness-dependent measurement while disregarding the influences of the container wall can be very advantageous.

In an embodiment the device has sensor materials and readout technology for measuring and controlling a cell culture process in a disposable bioreactor.

Further preferred applications result from single use process performances which are also based on enclosing the performed methods in plastic containers. Here similar applications are conceivable as in the disposable bioreactors.

Beside the applications in the field of biotechnology for biotechnological production of in particular chemical compounds, another field of application is the food industry, for example for the alcoholic fermentation in bags, which are inserted in large tanks in order to reduce cleaning costs and contamination, where the exclusion of oxygen is a further important goal.

The invention also includes the use of the device in the food industry.

In an embodiment the device is configured to conduct the radiation through a layer thickness which scatters to such a degree that at the used transmission path lengths the thickness change does not result in a change of the scattering angle distribution at the detector. As a result of the irradiation, radiation is transmitted through the flexible container. In the interior of the flexible container, i.e., between the walls of the container, the layer thickness can be changed by deformation of the walls. Thus it is possible to change the transmission path lengths—i.e., the path lengths of the light through the medium to be analyzed and with this through the contained substances in the container—by changing the thickness of the layer in the interior of the container.

The invention also includes a method for optical, non-invasive determination of the concentration or other parameters of contained substances in a flexible container. The method includes a step of shining light onto the flexible container. The method further includes the step of detecting an intensity weakening of the used radiation with different transmission path lengths at at least one wavelength or a wavelength range for each parameter to be detected. The method also includes a step of forming a quotient of the measurements of different thicknesses of the container wall in order to eliminate an influence of the concrete container wall at hand.

In an embodiment of the method, a wide-area irradiation and a wide-area detection are performed.

In an embodiment of the method, the irradiation is always performed through a layer thickness, which scatters to such a degree that at the used transmission path lengths the change in thickness does not cause a change of the scattering angel distribution at the detector.

The invention also includes a use of the method for determining blood parameters of blood, which is arranged in a closed blood conserve.

The invention also includes a use of the device for performing at least a part of the steps of the method.

### DESCRIPTION OF THE DRAWINGS

FIG. 1 shows a sequence of the detection of the measurement values. For a minimally required sequence, the sequence 100 shown in the center is to be performed. By means of the shown sequence 100, a parameter of a content substance can be detected. The term detection of a parameter in this context means the determining of a value for a parameter. For example the term detecting the concentration in the present context means a value of the concentration.

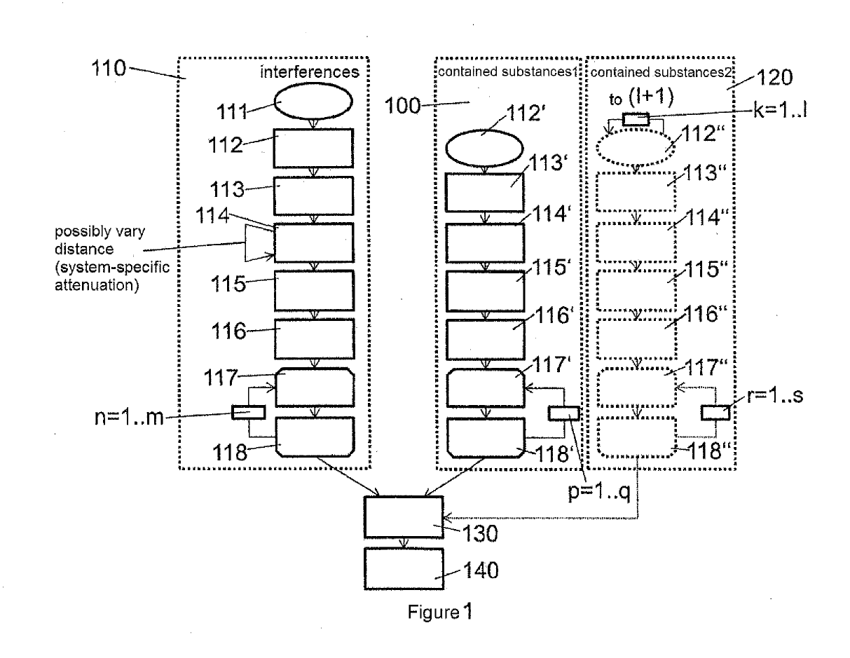

When a higher accuracy is required, the steps of the sequence 110 (interferences) shown on the left hand side are to be performed. For detecting further parameters the sequence 120 shown on the right hand side is to be performed for each parameter. Also parameters for other contained substances can be detected. This is possible by adjusting the wavelengths to be introduced in correspondence to the parameters of the other contained substances. For example concentrations of different contained substances of a flexible container can be detected. Thus with the shown method different parameters of a contained substance can be detected as well as a parameter of multiple contained substances. It is also possible to detect different parameters of multiple contained substances.

In the following the steps of the sequence 110 are explained.

In a step 111 the interferences are detected by measuring dark signals of the detector at turned off illumination and optionally with or without the sample in the beam path. In step 112 an irradiation with a wavelength or a wavelength range □₀ is transmitted through the transmission path through the sample, which transmission path is the same for all measurements, i.e., a wavelength which is more strongly absorbed by unknown weakenings than from the parameters to be determined in the content substances. Usually the wavelength or wave length range □₀ is selected so that it is in the vicinity of the □₁—or ε₂ ff—that are to be analyzed in order to enable a correction of the changed scatter angle distribution by the measuring at □₀.

In step 113 the device is adjusted to a starting distance d₀, and in step 114 the intensity of the radiation impinging on the detector is determined in the absence of the sample. This serves for compensating thickness-dependent, system-related intensity changes. In this way a reference value to a defined intensity is obtained for each thickness to be measured, to which intensity can later be normalized in order to eliminate these interferences. The elimination of the variable can also be accomplished by linear interpolation with only a few grid point of a measuring variable or in another appropriate manner without influencing the performance of the method according to the invention.

In step 115 the sample is introduced into the transmission path length so that in the subsequent step 116 a measurement I₂(ε₀, d₀) is performed, which detects an intensity at inserted sample and with influence of unknown weakenings at the starting thickness d₀. The subsequent steps 117 and 118 include successive changes of the thickness from d₀ to dₘ (n is here used as variable of the appropriate number of intermediate steps) (step 117) with measurements ε₂(ε₀, dₙ) (step 118) in order to detect the thickness dependent weakenings as raw values.

For detecting the parameter 1 of the contained substance, a similar sequence as for the interference factors is provided, with the sole difference that a different wave length or a different wavelength range ε1 is used, which is more strongly absorbed by the parameter 1 of the contained substance to be determined than by unknown weakenings or other parameters of the contained substance to be determined. The steps of sequence 100 that correspond to the steps of the sequence 110 are marked with a line. The variable for the appropriate number of intermediate steps is here designated p, the final thickness d_{q}. n and p (number of the measured thicknesses) may correspond to each other, however this is not strictly required.

The starting thickness or, when the process is performed in reverse order, the final thickness, however, should correspond. The order of the steps can also be changed.

For detecting further parameters 2 to (□+1) of the content substance or the content substances the same sequence is also provided. The steps of sequence 120 that correspond to the steps of sequence 110 are indicated with two lines. The variable I can be 1 or a different number. For each determined parameter at least one wavelength or a wavelength range □ₖ is selected, which is respectively absorbed more strongly by the respective parameter 2 to (□+1) of the content substance to be determined than by unknown weakenings or other parameters of the contained substance(s) to be determined. The variable for the appropriate number of intermediate steps is here designated r, the final thickness ds. r and n and/or p (number of the measured thicknesses) or s and m and/or q (final thickness) may correspond to each other, however this is not strictly required.

For the analysis in a step 130, after the described steps for normalization, elimination of the interferences and the response function R is applied to the raw values of the measuring variables Iₓ, whose input values are the wavelengths or wavelength ranges □_{z}, the associated weakened intensity values at a wavelength or a wavelength range and a thickness Iₓ(□₁, dₓ) and the thickness of the container through witch radiation is transmitted.

Without limiting the generality the concentration of a content substance is stated as result of the analysis (step 140). These can also be other parameters of the contained substance(s).

### LIST OF REFERENCE SIGNS

□₀ wave length or wavelength range, at which a weakening of the radiation as far as possible does not depend on the parameters of the content substances to be determined

□₁ wavelength or wavelength range at which a weakening of the radiation predominantly depends on the parameters of the content substance(s) to be determined

□ₖ wavelength or wavelength range, at which a weakening of the radiation of further parameters of the content substance (s) to be determined as far as possible does not depend n the other parameters of the content substance (s) to be determined, except when these serve for improving the accuracy

d transmission path length

I intensity

k variable for the number of the further wavelengths(ranges) for determining further parameters of the content substances or improved accuracy (starts at 2)

l number between 1 and the number of the further wavelengths (ranges) for determining further parameters of the content substances or improved accuracy

n variable for the thickness gradation of the transmission path length d at □₀

m number between 1 and the total number of the thickness gradation of the transmission path length at □₀

p variable for the thickness gradation of the transmission path length □₁

q number between 1 and the total number of the thickness gradation of the transmission path length d at □₁

r variable for the thickness gradation of the transmission path length d at each realized wave length or each realized wavelength range □ₖ

s number between 1 and the total number of the thickness gradation of the transmission path length d, optionally different at each realized wavelength or each realized wavelength range □ₖ.

X index/variable of unknown content

1.-15. (canceled)

16. A device for optical, non-invasive determination of a concentration or other parameter of a substance contained in a flexible container, said device comprising:
a light source, configured to radiate light onto the flexible container;
at least one detector, configured detect a weakening of an intensity of the radiation at at least one wavelength or a wavelength range of the radiation at different transmission path lengths of the radiation through the container; and
a processor configured to eliminate an influence of the concretely present container wall by forming a quotient of the measurements of different thicknesses.

17. The device of claim 16, wherein the light source is configured for a wide-area irradiation and the detector is configured for a wide-area detection.

18. The device of claim 16, wherein the device is configured to always conduct the radiation through a layer thickness, which scatters to such a degree that at a used transmission path length, a change in thickness does not result in a change of the scatter angle distribution at the detector.

19. The device of claim 16, wherein the device is configured to determine blood-parameters of blood, contained in a closed blood conserve.

20. The device of claim 19, wherein the device comprises a mechanical arrangement, which is configured to change a distance of two walls of the blood conserve in a region of a measurement field in a defined manner.

21. The device of claim 20, wherein the device is configured to determine a hemoglobin content of blood.

22. The device of claim 16, further comprising sensor materials and readout technology for measuring and controlling a cell culture process in a disposable bioreactor.

23. The device of claim 16, wherein the at least one detector is arranged relative to the light source so that a detected radiation travels different paths of different lengths through the substance in the container.

24. A method or optical, non-invasive determination of a concentration or other parameters of a substance contained in a flexible container, said method comprising:
irradiating the flexible container with light;
detecting a weakening of an intensity of the light at different transmission path lengths at at least one wavelength or a wavelength range for each parameter to be detected; and
forming a quotient of measurements of different thicknesses of the container wall, to eliminate an influence of the container wall.

25. The method of to claim 24, wherein a wide-area irradiation and a wide area detection are performed.

26. The method of claim 24, wherein the irradiation is always performed through a layer thickness, which scatters to a degree so that at a used transmission path length a change of thickness does not cause a change of a scatter angle distribution at the detector.

27. The device of claim 16, wherein the substance is blood contained in a closed blood conserve.

28. The method of claim 24, wherein the substance is blood contained in a blood conserve.

28. The device of claim 16, for use in the food industry.

30. A device for optical, non-invasive determination of a concentration or other parameter of a substance contained in a flexible container, said device comprising:
a light source, configured to radiate light onto the flexible container;
at least one detector, configured detect a weakening of an intensity of the radiation at at least one wavelength or a wavelength range of the radiation at different transmission path lengths of the radiation through the container; and
a processor configured to eliminate an influence of the concretely present container wall by forming a quotient of the measurements of different thicknesses,
said device being adapted to perform for performing at least part of the steps of the method of claim 24.
